(19) United States
(12) Patent Application Publication
(10) Pub. No.: US 2012/0253361 A1
(43) Pub. Date: Oct. 4, 2012

(54) Microdrive for Use in Stereotactic Surgery

(75) Inventors: Igor Drstvensek, Celje (SI); Tadej Strojnik, Race (SI)

(73) Assignee: ORTOTIP, LTD., Maribor (SI)

(21) Appl. No.: 13/503,795

(22) PCT Filed: Oct. 26, 2010
(86) PCT No.: PCT/SI2010/000060

§ 371 Date: Jun. 20, 2012

(30) Foreign Application Priority Data
Oct. 26, 2009 (SI) P-200900319

(51) Int. Cl. A61B 19/00

(52) U.S. Cl. 606/130

(57) ABSTRACT

Instrument (9) micro positioning device on the stereotactic system consists of the linear drive with the collet-chuck and the bracket (1) with the instrument (9) guide. The collet-chuck fixes and positions the instrument (9) with the collet (6a, 6′a) and associated taper-nut (7, 7′). The collet (6a) with the associated nut (7) is intended for clamping and positioning a single instrument, whilst the collet (6′a) with the associated taper-nut (7) is intended for the simultaneous securing and positioning of five instruments (9) or microelectrodes at a time. Through holes of the collet (6′a) are coaxial with bores of the central spacer as instrument positioning guide (9), where the sections of the collet (6′a) form and define the four through holes, which are symmetrical and evenly-spaced around the central through-hole.

### FIELD OF THE INVENTION

The object of this patent application relates to microdrives, i.e. devices for the safe and accurate placement of a surgical instrument into a portion of a patient's central nervous system of by a simple mechanical operation such as linear micro-positioning. Micro drives are mechanisms or devices for linear positioning, which enable the controlled movement of a medical instrument or other similar electro-technical accessories between two limiting positions. Such devices are used as mechanisms for implementing cinematic operations or interventions, where the exact positioning of the instrument is of crucial importance. Such an example is the use of a microdrive for the positioning of electrodes or electrode tubes during the stimulation of deep-brain nuclei or during stereotactic biopsy and the introduction of drainage catheters.

### BACKGROUND OF THE INVENTION

The technological problem addressed by this patent-application is the present lack of a device that is easy to use and handle, whilstmaintaining a minimal number of components thus ensuring exact positioning with the possibility of modularly fixing different medical instruments.

Stereotaxy is a precise surgery procedure for three-dimensional access to small targets inside the brain. A stereotactic system is used in order to perform stereotactic surgery. It consists of a stereotactic reference mechanism or stereotactic device and a unit for precise positioning within the Cartesian coordinate system.

A significant number of relevant solutions are listed in the International Patent Register. The following describes the solution, which describes the state of the art technology in this area.

According to U.S. Pat. No. 5,817,106A, the micro drive is installed on the stereotactic system, more precisely on the rigid coordinate ring using this measuring device for the implementation of reference positioning. According to the said patent application the micro-drive consists of a frame and a linear drive, which moves the actuation point of the instrument between two limiting positions, where the instrument is mounted on the plane guide using the spacer and associated positioning screw. The disadvantage of such a micro-drive is primarily its handling difficulties. This may cause complications during the neurosurgical process, due to the used components and securing of the instrument on the micro drive.

### SUMMARY OF THE INVENTION

Microdrive for use in stereotactic surgery consists of elements or components, such as a linear-drive and a spacer with a collet-chuck and a framework for the integrated guidance of the instrument. The linear-drive of the device moves a sliding-trolley, using the spacer and collet-chuck, between two limiting positions, between which the installed medical instrument is longitudinally positioned according to the instrument's axis, relative to the referenced stereotactic frame.

This device is primary intended for the introduction of electrodes for stimulating deep-brain nuclei. It should be understood, that such a device may also be used for other known and unknown surgical procedures and operations, as it enables the installation and micro-positioning of different medical instruments and accessories, included or otherwise in this patent application, and labelled as the generic term ‘instrument’.

A preferred embodiment of the present invention will now be described with reference to the accompanying drawings of microdrive for use in stereotactic surgery whereby, for clarity of the figures, the stereotactic system to which the microdrive is attached to is not shown.

### BRIEF DESCRIPTION OF THE DRAWINGS

FIG. 1 is an isometric view of the Microdrive for use in stereotactic surgery, where the device includes a securing system for positioning of a single medical instrument. The following items are shown and marked on FIG. 1: bracket (1), guide-bar (2), button (3), sliding trolley, i.e. slide with an integrated threaded-nut (5), spacer (6), taper-nut (7), central spacer (8) and the instrument (9).

FIG. 2 is an isometric view of the single instrument securing system (9) also known as a spring-collet, where the elements are shown in the dismounted position. The following items are shown and marked in FIG. 2: spacer (6) with an integrated spring-collet (6a), the associated taper-nut (7), and the collet-bracket (6b).

FIG. 3 is a frontal view of the Microdrive for use in stereotactic surgery, where the device includes the securing system for positioning of a single medical instrument (9). The following items are shown and marked in FIG. 3: plane A-A, bracket (1), guide-bar (2), button (3), threaded-nut (5), spacer (6), and the instrument (9).

FIG. 4 is a transverse cross-section taken along line A-A of FIG. 3. The following items are shown and marked in FIG. 4: bracket (1), guide-bar (2), button (3), threaded-spindle (4), threaded-nut (5), spacer (6), taper-nut (7), central-spacer (8) and the instrument (9). Detailed view B and detailed view C are further defined in FIG. 4.

FIG. 5 is a detailed view B of the securing system for clamping and positioning one sole instrument (9), fixed into the collet-chuck. Furthermore, the elements in FIG. 5 are shown in a closed-state, which means that the taper-nut (7) is tightened around the tapered-neck of the collet-chuck and, therefore, the instrument (9) is then attached, i.e. secured relative to the positioning system. The following items are shown and marked in FIG. 5: spacer (6), taper-nut (7) and the instrument (9).

FIG. 6 is a detailed view C of the securing system for clamping and positioning one sole instrument (9), whereby the instrument (9) is inserted into the collet-chuck, which means that the spacer (6) with the associated taper-nut (7) serves as the instrument (9) positioning guide-bar. Furthermore, the elements in FIG. 6 are shown in open-state, which means that the taper-nut (7) is slack and, therefore, the instrument (9) is free to move along its axis relative to the positioning system. The following items are shown and marked in FIG. 6: spacer (6), taper-nut (7), and the instrument (9).

FIG. 7 is an isometric view of the Microdrive for use in stereotactic surgery, where the device includes a multi-object securing system for the repositioning and securing of, in this case, five medical instruments (9). The following items are shown and marked in FIG. 7: bracket (1), guide-bar (2), button (3), threaded-nut (5), spacer (6′), taper-nut (7′), central-spacer (8′) and the plurality of the instrument (9).

FIG. 8 is an isometric view of the multi-object securing system where elements are shown in the dismounted position. Disclosed multi-object split, i.e. the spring-type collet-chuck is hereby performed as a five-instruments (9) positioning system, where its elements are marked as follows: spacer (6′) with an integrated multi-object spring collet (6′a), acting as a compressible tapered-neck with associated taper-nut (7′), and the collet-bracket (6′b).

FIG. 9 is the frontal view of the Microdrive for use in stereotactic surgery with an integrated multi-object securing system for the positioning of five medical instruments (9) at a time. The following items are shown and marked in FIG. 9: plane D-D, bracket (1), guide-bar (2), button (3), spacer (6′) and the plurality of the instrument (9).

FIG. 10 is a transverse cross-section taken along the line D-D of FIG. 9. The following items are shown and marked in FIG. 10: bracket (1), guide-bar (2), button (3), threaded-spindle (4), threaded-nut (5), spacer (6′), taper-nut (7′), central-spacer (8′) and the plurality of the instrument (9). Detailed views E and F are further defined in FIG. 10.

FIG. 11 is the detailed view E of the multi object securing system, where the instruments (9) are fixed into the collet-chuck. Furthermore, the elements of the device in FIG. 11 are shown in a closed-state, which means that the taper-nut (7′) is tightened around the tapered-neck of the multi-object spring-type collet (6′a) and therefore, the instruments (9) are attached, i.e. fixed relative to the multi instruments (9) positioning system. The following items are shown and marked in FIG. 11: spacer (6′), taper-nut (7′), and the plurality of the instrument (9).

FIG. 12 is the detailed view F of the multi-object securing system, where the elements in FIG. 12 are shown in open state, which means that the taper-nut (7′) is slack, i.e. released and the instruments (9) are therefore free to move along its axes relative to and through the multi-instrument (9) securing system. The following items are shown and marked on the FIG. 12: spacer (6′), taper-nut (7′), and the plurality of the instrument (9).

### DETAILED DESCRIPTION OF PREFERENTIAL EMBODIMENT

To facilitate the handling of the device and the cleaning and sterilization of the device's components for surgical activities, the framework concept of the device within an integrated linear drive is designed in such a way, that the instrument's (9) guide-bar is a segment of the bracket (1) by which the device is attached, i.e. mounted or fixed onto the frame of the stereotactic system.

In the preferential embodiment, the three-point instrument (9) guidance system consists of the front spacer (6, 6′) on the bracket (1) with an associated taper-nut (7, 7′), the central spacer (8, 8′) and the rear spacer (6, 6′) with associated taper-nut (7, 7′) mounted on the slides of the threaded nut (5).

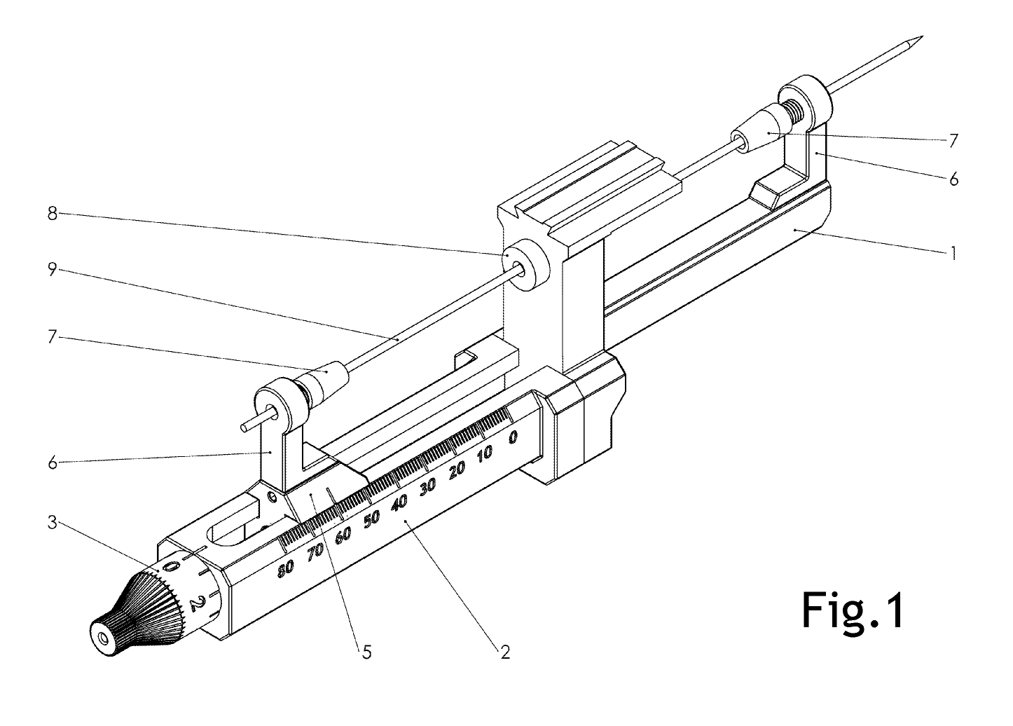

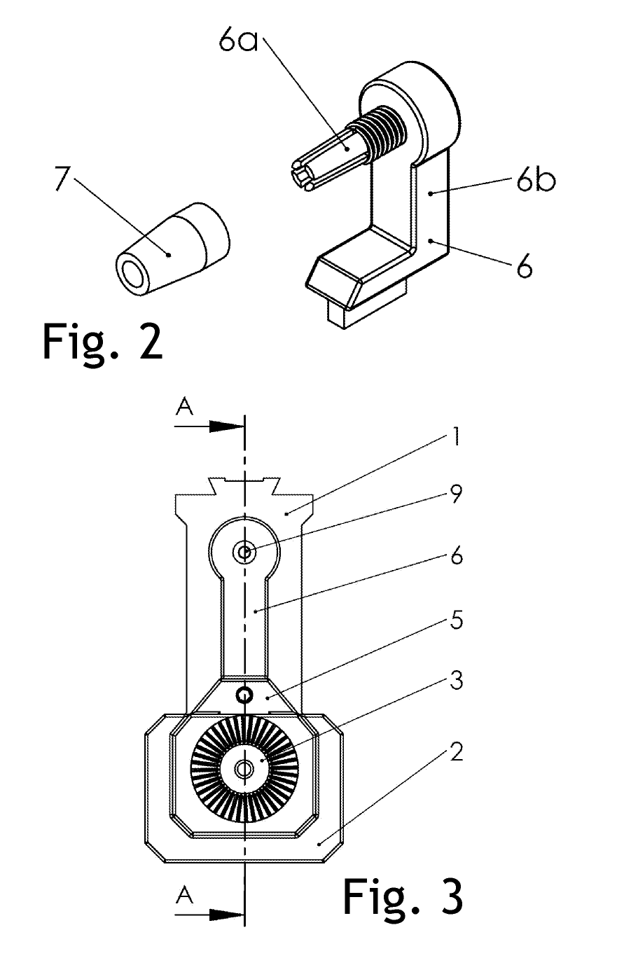

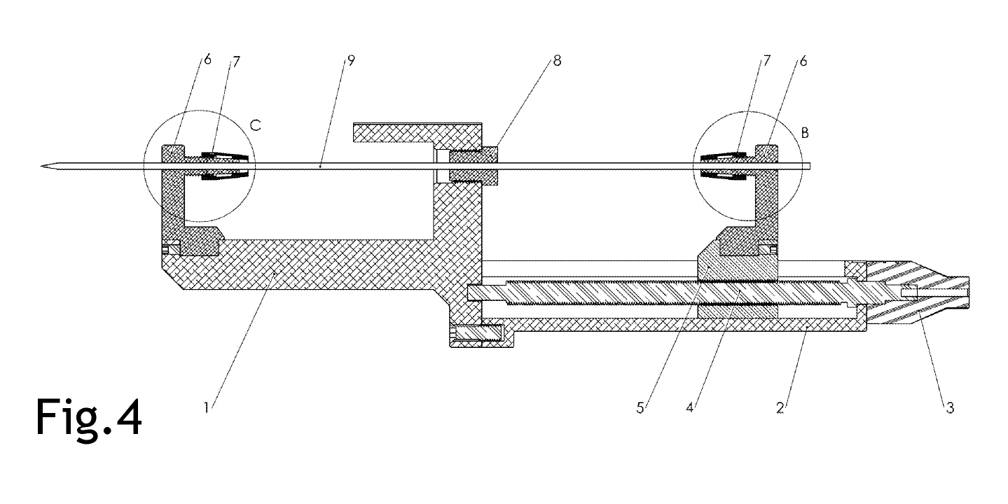

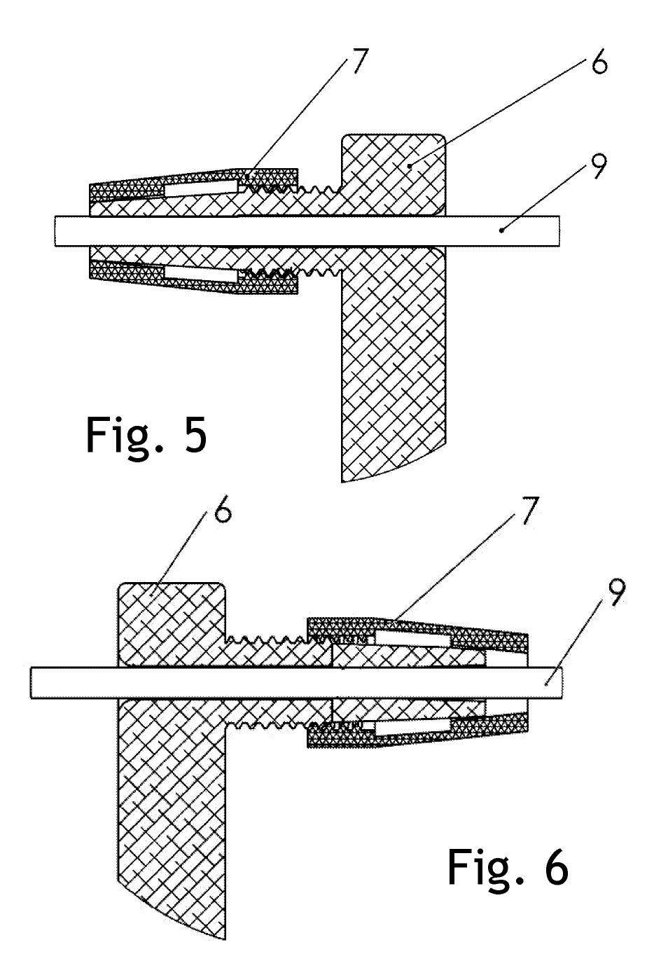

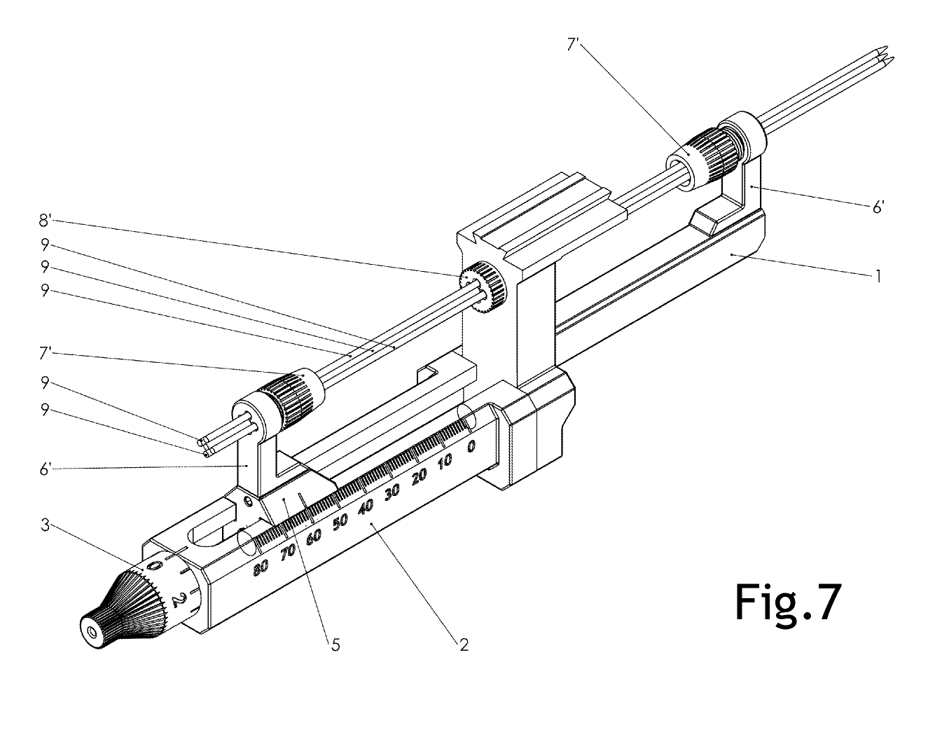

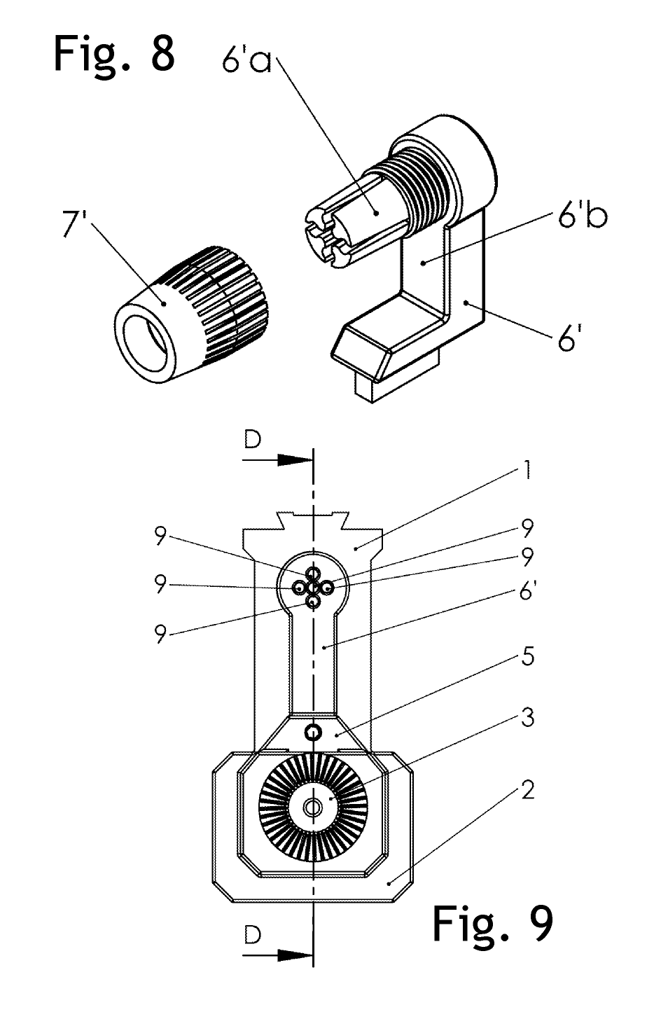

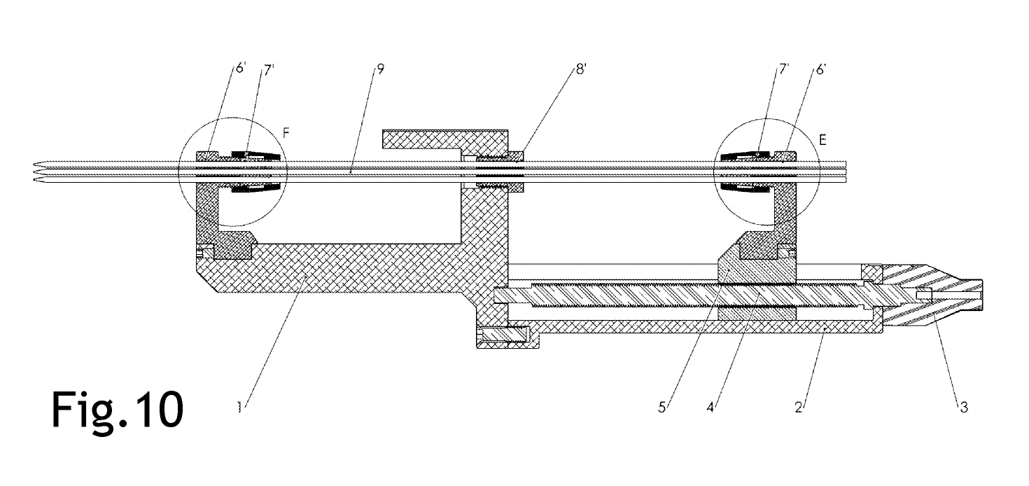

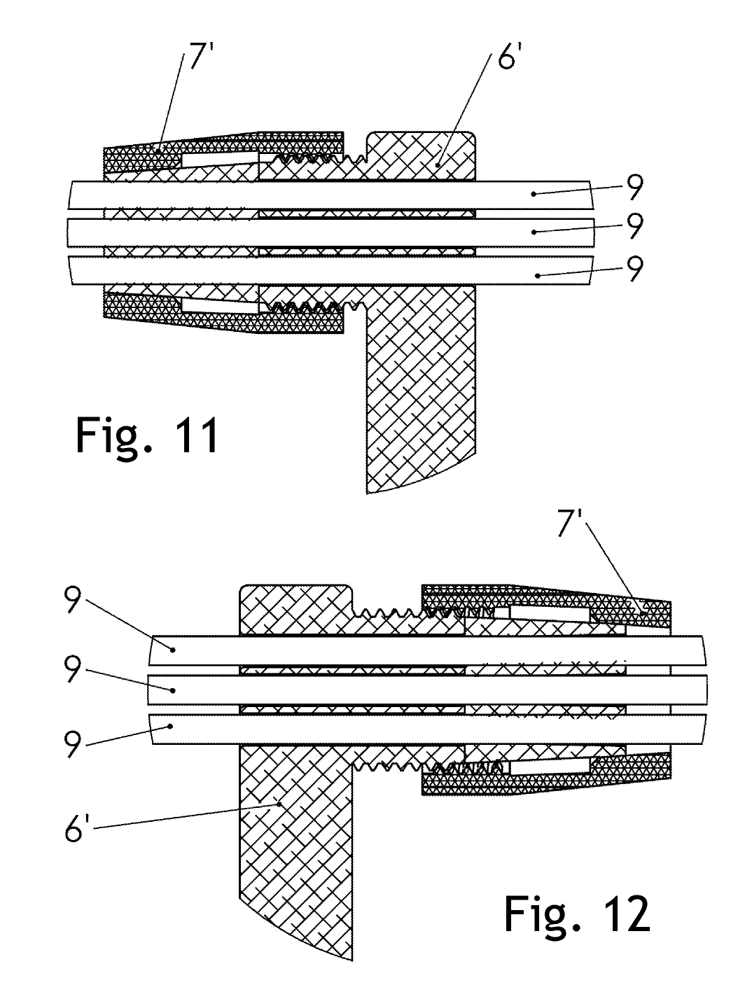

The guidance system may comprise different compatible securing systems, i.e. spacers (6, 6′) or collets (6a, 6′a), which enable one or multiple instruments (9) to be positioned simultaneously. More precisely, the Microdrive where used in stereotactic surgery is hereby disclosed according to two different variations. The first variation, as shown in FIGS. 1-6, where the elements of the device are designed for only one instrument's (9) positioning; therefore the device comprises front and rear spacers (6) with associated nuts (7) and a central spacer (8), where each of the listed elements has one coaxial through-hole for positioning and clamping the instrument (9). In the second variation shown in FIGS. 6-12 the device is meant for positioning and clamping five instruments (9) at a time; therefore the Microdrive for use in stereotactic surgery comprises front and rear spacers (6′) with associated taper nuts (7′), and the central spacer (8′).

In the preferred embodiment of the device, i.e. Microdrive for use in stereotactic surgery comprises a three point guidance system for repositioning the instrument (9) along its axis, relative to the device installed in the stereotactic system and furthermore the device has a linear drive with an adopted sliding-trolley with an integrated actuating securing system, i.e. spring-collet chuck for instrument (9) insertion, and the clamping and positioning of the instrument (9) between two extreme positions.

The linear-drive in the preferential embodiment is designed as a threaded engagement means, more precisely as a threaded-spindle (4) with an appurtenant threaded nut (5) integrated into the slides, which is based on the principle of a threaded-spindle moving within a guide groove (2). It should be understood, that the rotation of the button (3) is transformed into a linear movement of the slides along the guide-groove (2) between two extreme positions. The preferential pitch of the threaded-spindle is 1 mm, which ensures ease of use and a precise positioning at the micro-level. The button (3) is designed as a two-level device with different diameters, which enables quick positioning when rotating around a smaller diameter and very precise, micro-positioning when rotating around outer side, i.e. over a bigger diameter.

Furthermore, the linear drive of Microdrive for use in stereotactic surgery comprises at least one securing system, i.e. a clamping apparatus well known as a spring collet-chuck, which is movable along a guide-groove (2) between two extreme positions. As a preferential embodiment, the Microdrive for use in stereotactic surgery comprises a three-point guidance system containing a concentrically-aligned front-spring collet-chuck, central-guidance element, and a rear collet-chuck, where the front and rear collet-chucks may be identical, but have reverse oriented elements.

As a preferential embodiment, the spring-collet (6a, 6′a) is designed as slotted and tapered bushing, and when the tapered-nut (7, 7′) is tightened, i.e. slipped over the slotted and tapered bushing, the slots of the collet (6a, 6′a) tend to close and the bushing thereby grips the instrument (9) into place. It can be understood, that the collet (6a, 6′a) can be designed and manufactured as one body-part, such as a spring-type collet and, on the other hand, the collet (6, 6′a) can also be designed and manufactured as a multi body-part element, where individual slots are assembled, using elastic ring, the into integral collet (6a, 6′a)-type bushing. It should be understood, that slots from bushing form at least one through-hole with a nominal diameter for the accompanying instrument (9) securing. Furthermore, for the preferential embodiment of Microdrive in stereotactic surgery, the collet (6′a) of the multi-object securing system is manufactured and performed as an assembled multi-body-part collet (6′a), where individual segments of the collet (6′a) generate at least two, preferably five, through holes with nominal diameters for positioning of the accompanying instrument (9).

It should be understood, that the spacer (6, 6′) may be designed in many variants. For example; the collet bracket (6b, 6′b) of the front spacer (6, 6′) can be integrated into the bracket (1) and in this case only collets (6a, 6′a) of the collet-chuck are to be exchanged. In accordance with the previous statement, the collet (6a, 6′a) can be installed into or onto the collet-bracket (6b, 6′b) with the junction, where the through-holes of each individual collet (6a, 6′a) and the through-holes of a central spacer (8, 8′) are in the coaxial position, according to the required and predefined positions of the instruments (9). In this way the modified collet (6a, 6′a) is then fixed on the collet bracket (6b, 6′b) using an appropriate element, such as a nut, a detent or a snap-ring. The same applies for the rear spacer (6, 6′), which can also be integrated into the slides of the linear drive or central spacer (8, 8′), which can also be fixed on the bracket (1) using the appropriate junction or element. The through-holes of the collet (6a, 6′a) and the through holes of the central spacer (8, 8′) are primarily intended for the manipulation of cylindrical instruments (9) with external diameters of 1 mm, 1.27 mm, 1.4 mm, 1.65 mm and 1.88 mm. In accordance with that described above, the collet (6a, 6′a) through-holes diameters and the central spacers (8) through-holes diameters equal the determined diameters, when considering the tolerance range, i.e. the clamping capacity which is, preferably at least 10% of the nominal diameter of the instrument (9). It should be understood that by the threading of, i.e. tightening the taper-nut (7, 7′) on the appropriate collet (6a, 6′a), the spring-collet elements fix the instrument (9) in place. During this process, the determined tolerance range, i.e. the clamping capacities of each individual through hole are neutralized and the equivalent friction force between the collet sections (6a, 6′a) and the instrument (9) is established. The collet sections (6a, 6′a) are generally placed symmetrically around the central axis, where the segments form four through-holes on the collet (6′a), symmetrically-placed around the central through-hole, which is formed by the collet-sections (6′a).

According to another aspect of the presented invention, a method is provided for operating a Microdrive in stereotactic surgery, characterized by the following steps:
installing the instrument (9) to the device by inserting it through the securing system, whereby the collet-chuck has at least one through-hole that is coincidental with the instrument (9) guidance system
clamping the instrument (9) to the securing system, i.e. collet-chuck by the spring-collet (6a, 6′a) which is preferably designed as an slotted and tapered bushing, and when the tapered nut (7, 7′) is tightened, i.e. slipped over the slotted and tapered bushing, the slots of the collet (6a, 6′a) tends to close and the bushing thereby grips the instrument (9) into place.
positioning the instrument (9) between two extreme positions of the microdrive for use in stereotactic surgery, where the instrument is fixed in place by the said collet-chuck.

### INDUSTRIAL USE

This invention finds utility and applicability within the surgical appliance field. The presented Microdrive for use in stereotactic surgery is primarily intended for the performance of neurosurgical procedures or operations. The disclosed instrument's (9) securing method using a single-spindle collet (6a) is intended for surgical procedures and tissue investigations or procedures, related to tissue biopsy, where by appropriate modifications, other medical or electrical devices may be also fixed on the single-spindle collet, for which exact positioning is essential (Such as the introduction of drainage catheters for brain partitions or cystes). The system of parallely fixing five instruments (9) by using a multiple, i.e. five-spindle collet (6′a) and a securing taper-nut (7′) is primary intended for neurosurgical processes within the range of stimulating of deep brain-nuclei, also known as Deep Brain Stimulation (DBS) and for Micro Electrode Recording (MER).

1. Microdrive for use in stereotactic surgery comprising a framework with adapted linear-drive to operatively engage a securing system, which is movable between two positions for the clamping and positioning of at least one instrument (9) characterized in that the said securing system of Microdrive for use in stereotactic surgery includes a through-hole collet-chuck type clamping apparatus, where the collet-chuck includes a holding device that forms a collar around the at least one instrument (9) and exerts a clamping force on the instrument (9) when the collet is squeezed and tightened via an outer one body part collar, whereby the segments of the collet forms at least one through-hole for the inserting, clamping, and positioning of the at least one instrument (9) between the two positions.

2. Microdrive for use in stereotactic surgery as, in claim 1, characterized in that said segments form five through-holes for the insertion, clamping, and positioning of five instruments (9) at a time; wherein one of the through-holes is co-axial with a central-axis of the collet, and where four of the through-holes symmetrically placed about the central-axis.

3. Microdrive for use in stereotactic surgery as in claim 1 characterized in that said collet is a spring collet insert for clamping the at least one instrument (9) in place, whereby the spring collet (6a, 6′a) is includes a slotted and tapered bushing, and when the collar (7, 7′) is slipped over the slotted and tapered bushing, the slots of the collet (6a, 6′a) close to secure the at least one instrument (9).

4. Microdrive for use in stereotactic surgery as in claim 1 characterized in that said collet is a collet chuck insert, wherein said plurality of segments are assembled with elastic ring into integral collet (6a, 6′a) having a slotted and tapered bushing, wherein when the collar (7, 7′) is slipped over the slotted and tapered bushing, the slots of the collet (6a, 6′a) close and the bushing thereby secures the instrument (9) in place.

5. Microdrive for use in stereotactic surgery as in claim 1 characterized in that that framework of Microdrive for use in stereotactic surgery further comprise three point guidance system, comprising the concentrically aligned front spring collet chuck, central guidance element and rear collet chuck; and furthermore the three point instrument (9) guidance system comprise the front spacer (6, 6′) on the bracket (1) with associated taper nut (7, 7′), the central spacer (8, 8′) and the rear spacer (6, 6′) with associated taper nut (7, 7′) mounted on the sliding trolley being part of the linear drive, whereby the framework of the device with adapted linear drive is mounted or fixed on the frame of the stereotactic system.

6. Microdrive for use in stereotactic surgery as in claim 1 characterized in that that said linear drive includes a threaded engagement means and appurtenant threaded nut (5) integrated into a sliding trolley the threaded engagement means is arranged in a guide groove (2), a button is coupled to one end of the threaded engagement means such that rotation of the button (3) is transformed into the linear movement.

7. Microdrive for use in stereotactic surgery as in claim 6 characterized in that that said button (3) is designed as a two level device with different diameters for a first level of positioning when rotating on smaller diameter and a micro positioning when rotating on an outer diameter.

8. Microdrive for use in stereotactic surgery as in claim 1 characterized in that said through hole is characterized by nominal diameter of instrument (9), where the preferential nominal diameter of instrument (9) is selected from a group comprising: 1 mm; 1.27 mm; 1.4 mm; 1.65 mm; 1.88 mm.

9. A method of operating Microdrive for use in stereotactic surgery, characterized by steps of:
installing the instrument (9) to the device by inserting it through a securing system, whereby the collet-chuck has at least one through-hole that is coincidental with the instrument's (9) guidance system;
clamping instrument (9) to the securing system with a collet-chuck having a slotted and tapered bushing and a tapered-nut, and when the tapered-nut (7, 7′) is tightened the slots of the collet (6a, 6′a) close and the bushing thereby grips the instrument (9) into place;
positioning the instrument (9) between two positions for stereotactic surgery, where the instrument is fixed in place by said collet chuck.

10. A microdrive for use in stereotactic surgery comprising:
a guide bar;
a linear drive coupled to the guidebar;
at least one instrument operably coupled to the linear drive;
a first collet operably coupled to the linear drive, the first collet having a plurality of segments movable between a first position and a second position, the plurality of segments forming a first hole sized to receive the at least one instrument; and,
a collar operably coupled to the first collet and configured to move the plurality of segments from the first position to the second position, wherein the at least one instrument is secured to the linear drive when the plurality of segments are in the second position.

11. The microdrive of claim 10 wherein the plurality of segments are configured to form four second holes disposed symmetrically about the first hole.

12. The microdrive of claim 10 wherein the first collet includes a slotted and tapered bushing operably coupled to the collar, the slots being arranged to move to a closed position to secure the at least one instrument in response to movement of the collar.

13. The microdrive of claim 12 wherein the collar is a tapered nut.

14. The microdrive of claim 10 wherein the first collet further includes a plurality of slots formed between the plurality of segments when in the first position and an elastic ring disposed about the segments.

15. The microdrive of claim 10 further comprising:
a second collet concentrically aligned with the first collet;
wherein the linear drive includes a bracket having a spacer on one end, the second collet being operably coupled to the spacer.

16. The microdrive of claim 15 wherein the guide bar includes a groove, the linear drive being operably coupled to the groove.

17. The microdrive of claim 16 wherein the linear drive comprises:
a spindle rotationally coupled to the framework within the groove and coupled to the bracket on one end, the spindle having a thread pitch of about 1 millimeter; and,
a button coupled to the spindle opposite the bracket.

18. The microdrive of claim 10 wherein the diameter of the instrument is less than 1.88 millimeters.

19. The microdrive of claim 10 wherein the diameter of the instrument is between 1 millimeter and 1.65 millimeter.

20. The microdrive of claim 10 wherein the diameter of the instrument is selected from a group comprising: 1 millimeter; 1.27 millimeter; 1.4 millimeter; 1.65 millimeter; and 1.88 millimeter.
